(19) United States
(12) Patent Application Publication
(10) Pub. No.: US 2018/0340008 A1
(43) Pub. Date: Nov. 29, 2018

(54) ANTIBIOTIC OLIGOPEPTIDE MIMETICS

(71) Applicant: BIOXINESS PHARMACEUTICALS, INC., Hercules, CA (US)

(72) Inventors: Mansour BASSIRI, Hercules, CA (US); Richard D. GLESS, Hercules, CA (US)

(21) Appl. No.: 15/987,336

(22) Filed: May 23, 2018

Related U.S. Application Data
(60) Provisional application No. 62/510,352, filed on May 24, 2017.

(51) Int. Cl. C07K 5/065; C07C 237/04; C07C 323/25; C07K 5/078; A01N 43/38; A01N 37/18; A61P 31/04

(52) U.S. Cl. CPC C07K 5/06078; C07C 237/04; C07C 323/25; A61K 38/00; A01N 43/38; A01N 37/18; A61P 31/04; C07K 5/06156

(57) ABSTRACT

Disclosed are amino acid mimetics that possess antibiotic properties in prokaryotic cells. These mimetics are coupled to one or more optionally substituted amino acids provided that at least one of the amino acids is an optionally substituted amino acid selected from the group consisting of phenylglycine, tryptophan, phenylalanine, histidine, and tyrosine.

### FIELD OF THE INVENTION

This invention is directed to oligopeptides containing one or more amino acid mimetics. The amino acid mimetics employed with said oligopeptides possess intracellular antibiotic properties in prokaryotic cells. These mimetics are coupled to one or more optionally substituted amino acids provided that at least one of the amino acids is an optionally substituted phenylglycine, tryptophan, phenylalanine or tyrosine. Accordingly, disclosed are compounds, compositions and methods for treating a prokaryotic infection in a mammal as well as prodrugs for such compounds.

### STATE OF THE ART

Prokaryotic cells such as bacteria import amino acids by specific transporters that include those that transport oligomeric peptides. These transporters have been utilized in U.S. Pat. No. 8,580,859 to incorporate antibiotic methionine mimetics into bacteria. As disclosed, such mimetics are coupled to generic classes of natural and unnatural amino acids optionally substituted with a large number of substituents to provide for oligopeptides. Once taken up/transported into bacteria, the methionine mimetic prevents proper peptide synthesis by these bacteria resulting in bacterial death.

These compounds provided efficacious results as measured by in vitro assays indicating acceptable levels of intrabacterial methionine mimetics. However, significantly greater antibacterial properties for such oligopeptides would be advantageous as such would lead to greater and more rapid bacterial death.

### SUMMARY OF THE INVENTION

This invention is based, in part, on the discovery that certain specific L-amino acids provide significantly improved oligopeptide uptake by prokaryotic cells when attached to one or more amino acid mimetics. Once internalized, these oligopeptides are converted to their corresponding single amino acid and amino acid mimetic components by, for example, enzymatic processes. The mimetic then binds with high specificity to the tRNA^{AA} synthesase thereby inhibiting the natural amino acid from incorporating into the protein being synthesized by the bacteria, which leads to bacterial growth inhibition and cell death.

In one of its compound aspects, this invention provides for oligopeptide mimetics having from 1 to 9 optionally substituted amino acids and a C-terminal amino acid mimetic of formula I:

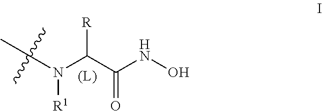

where:

R is selected from hydrogen, optionally substituted aryl, or optionally substituted C₁-C₆ alkyl wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;

R¹ is hydrogen or together with R forms an optionally substituted pyrrolidinyl ring wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;

provided that at least one of the optionally substituted amino acids is an optionally substituted aromatic amino acid selected from the group consisting of optionally substituted phenylglycine, optionally substituted phenylalanine, optionally substituted tyrosine, optionally substituted histidine and optionally substituted tryptophan; and

(L) indicates an L isomer at that stereochemical center;

including pharmaceutically acceptable salts and/or solvates thereof.

In one embodiment, this invention is directed to oligopeptide mimetics having from 1 to 9 optionally substituted amino acids and a C-terminal amino acid mimetic of formula II:

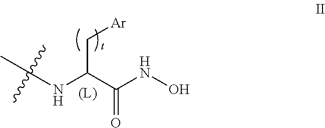

where:

Ar is an optionally substituted phenyl, napthyl, imidazolyl or indolyl group;

t is zero, one, or two;

wherein said optional substitution on said phenyl, napthyl, imidazolyl, or indolyl groups is from 1 to 3 substituents selected from the group consisting of C₁-C₄alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy; and

(L) indicates an L isomer at that stereochemical center;

including pharmaceutically acceptable salts and/or solvates thereof. In one embodiment for this oligopeptide mimetic, the oligopeptide mimetic has 1 or 2 optionally substituted amino acids, Ar is an optionally substituted phenyl or indolyl group; and/or t is zero or one.

In one embodiment, this invention is directed to oligopeptide mimetics having from 1 to 9 optionally substituted amino acids and a C-terminal amino acid mimetic of formula III:

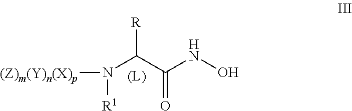

where m, n and p are independently 0 or 1;

X, Y and Z are each independently an L-isomer of an optionally substituted amino acid provided that at least one of X, Y and Z is an optionally substituted aromatic amino acid selected from the group consisting of optionally substituted phenylglycine, optionally substituted phenylalanine, optionally substituted tyrosine, optionally substituted histidine, and optionally substituted tryptophan;

wherein said optional substitution on said amino acids is from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄alkoxy;

R is selected from hydrogen, optionally substituted aryl, or optionally substituted C₁-C₆ alkyl wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;

R¹ is hydrogen or together with R forms an optionally substituted pyrrolidinyl ring wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;

(L) indicates an L isomer at that stereochemical center;

including pharmaceutically acceptable salts and/or solvates thereof.

In one embodiment, there is provided a compound of formula IV:

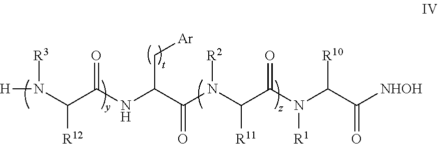

wherein:

R¹⁰, R¹¹, and R¹² are independently an optionally substituted side chain of a non-proline amino acid;

alternatively any one of R¹⁰ and R¹, R¹¹ and R² and/or R¹² and R³ forms an optionally substituted pyrrolidinyl group;

Ar is optionally substituted phenyl, optionally substituted histidyl, or optionally substituted indolyl;

t is 0, 1, or 2; and

y and z are independently 0, 1, 2, 3, 4, 5, 6 or 7 provided that the total of y plus z is no greater than 8;

wherein said optional substitution on said amino acids is from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄alkoxy;

including pharmaceutically acceptable salts and/or solvates thereof. In one embodiment, for this compound, t is zero or one, Ar is an optionally substituted phenyl or indolyl group; and/or the total of y plus z is not greater than 2.

In one embodiment, R is selected from the group consisting of hydrogen, phenyl, (R²⁰)ₐ-substituted phenyl, C₁-C₆ alkyl, and (R²¹)_{b}-substituted-(C₁-C₆ alkyl) where

R²⁰ is selected from the group consisting of hydroxyl, C₁-C₄ alkyl, C₁-C₄ alkoxy, halo, thiol, amino, nitro, cyano, and carboxy;

R²¹ is selected from the group consisting of hydroxyl, C₁-C₄ alkyl, C₁-C₄ alkoxy, halo, thiol, amino, amido, nitro, cyano, carboxy, aryl, substituted aryl, heteroaryl, substituted heteroaryl, guanidino, substituted guanidino, and C₁-C₄ alkylthiol; and

a and b are integers of from 1 to 3.

In one embodiment, this invention provides for a pharmaceutical composition comprising a pharmaceutically acceptable carrier and a compound of any of formula I-IV.

This invention also provides for bacteria comprising within it intracellular space a compound selected from any one of formula I-IV.

In one of its method aspects, this invention is directed to a method for killing prokaryotic cells which method comprises administering to said cells a compound selected from any one of formula I-IV above.

In one embodiment, the prokaryotic cells are bacterial cells.

In one embodiment, the bacterial cells are E. coli bacteria.

In one embodiment, this invention provides for a method of treating a subject with a bacterial infection which method comprises administering to the subject an effective amount of a compound selected from any one of formula I—IV or a pharmaceutical composition comprising an effective amount of a compound selected from any one of formula I-IV.

Representative compounds of this invention include the following as provided in Table I, for formula III, below as well as their salts and/or solvates:

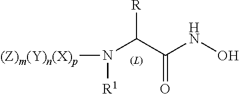

| Comp. | | | | | |
|---|---|---|---|---|---|
| No. | R | R¹ | X | Y | Z |
| 1 | Phenyl | H | Tyrosine | n = 0 | m = 0 |
| 2 | Benzyl | H | Phenylglycine | Alanine | Alanine |
| 3 | 4-Hydroxyphenyl | H | Glycine | Serine | Proline |
| 4 | Methyl | H | Tyrosine | n = 0 | m = 0 |
| 5 | Hydroxyl | H | Tryptophan | Phenylalanine | Serine |
| 6 | 3-Methylpropyl | H | Methionine | Phenylalanine | Cysteine |
| | (leucine side chain) | | | | |
| 7 | Hydroxymethyl | H | Proline | Phenylalanine | m = 0 |
| 8 | Thiomethyl | H | Phenylalanine | n = 0 | m = 0 |
| 9 | R and R¹ = pyrrodinyl | | Tyrosine | Phenylalanine | Glycine |
| 10 | Benzyl | H | Hydroxyglycine | Threonine | Alanine |
| 11 | 4-Hydroxybenzyl | H | Hydroxyglycine | Isoleucine | Aspartic acid |
| 12 | Cysteine | H | 2-amino-3-hydroxy- | Lysine | Phenylalanine |
| | | | 3-phenyl-proprionic | | |
| | | | acid | | |
| 13 | R and R¹ = pyrrodinyl | | Arginine | Cysteine | Tyrosine |
| 14 | Methoxy | H | Phenylalanine | Histidine | Glutamine |
| 15 | 2-Naphthyl | H | Phenylalanine | Valine | Leucine |
| 16 | 3-Indolylmethyl | H | Glycine | Glycine | m = 0 |
| 17 | 3-Indolylmethyl | H | Histidine | Lysine | Glycine |
| 18 | 4-Amino-n-butyl | H | Proline | Tyrosine | Tyrosine |
| 19 | Methoxymethyl | H | Phenylalanine | n = 0 | m = 0 |
| 20 | Ethylthiomethyl | H | 2-amino-3-ethoxy | Phenylalanine | m = 0 |
| | | | proprionic acid | | |
| 21 | R and R¹ = pyrrodinyl | | 2-amino-3-hydroxy- | Valine | Alanine |
| | | | 3-phenyl-proprionic | | |
| | | | acid | | |
| 22 | Phenylalanine | H | 2-amino-3-ethoxy | Leucine | Guanidine |
| | | | proprionic acid | | |
| 23 | Phenylalanine | H | Phenylalanine | Phenylanine | m = 0 |

### BRIEF DESCRIPTION OF THE DRAWING

FIGS. 1A and 1B demonstrates the superior antibacterial properties of an aromatic amino acid, phenylalanine, attached to a methionine mimetic as compared to glycine attached to the same methionine mimetic.

### DETAILED DESCRIPTION OF THE INVENTION

This invention provides for compounds and methods for killing prokaryotic cells and, in particular, pathogenic bacterial cells. However, prior to addressing this invention in more detail, the following terms will be defined.

### 1. DEFINITIONS

As used herein, the following definitions shall apply unless otherwise indicated. Further, if any term or symbol used herein is not defined as set forth below, it shall have its ordinary meaning in the art.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

Generally, reference to a certain element such as hydrogen or H is meant to include all isotopes of that element. For example, if an R group is defined to include hydrogen or H, it also includes deuterium and tritium. Compounds comprising radioisotopes such as tritium, C¹⁴, P³² and S³⁵ are thus within the scope of this invention. Procedures for inserting such labels into the compounds of this invention will be readily apparent to those skilled in the art based on the disclosure herein.

The term “oligomeric” refers to peptides having from 2-9 amino acids exclusive of the methionine mimetic of Formula I. In one embodiment, the oligomeric peptides are from 2 to 8, or from 2 to 4 amino acids exclusive of the methionine mimetic of Formula I. In one preferred embodiment, the amino acids in the oligopeptide do not include methionine.

The term “amino acid” refers to L-amino acids inclusive of all natural amino acids including 4-hydroxyproline, 5-hydroxylysine, and phenylglycine. Aromatic amino acids include phenylglycine, tryptophan, tyrosine, phenylalanine and the like.

The term “substituted amino acids” preferably refers to L-amino acids having 1 to 3 substituents on the amino acid side chain which substituents are selected from the group consisting hydroxyl, oxo, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substi-tuted heteroaryl, heterocyclic, substituted heterocyclic, thiol, C₁-C₄ thioalkyl, sulfonyl, amidino, amido, carboxyl, C₁-C₄ alkoxy, C₃-C₇ cycloalkyl, and C₁-C₄ alkyl-C₁-C₄ alkoxy.

“Alkyl” refers to monovalent saturated aliphatic hydrocarbyl groups having from 1 to 4 carbon atoms and preferably 1 to 2 carbon atoms. This term includes, by way of example, linear and branched alkyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), iso-butyl ((CH₃)₂CHCH₂—), sec-butyl ((CH₃)(CH₃CH₂)CH—), t-butyl ((CH₃)₃C.

“Substituted alkyl” refers to an alkyl group substituted with 1 to 3 substituents selected from the group consisting of hydroxyl, nitro, acyl, acylamino, aryl, substituted aryl, amino, C₁-C₄ alkylamino, di(C₁-C₄ alkyl)amino, cyano, halo, heteroaryl, substituted heteroaryl, heterocyclic, substituted heterocyclic, thiol, C₁-C₄ thioalkyl, amidino, amido, carboxyl, C₁-C₄ alkoxy, oxo, and C₃-C₇ cycloalkyl. In one embodiment, the substituted alkyl is a C₁-C₄ haloalkyl having from 1 to 3 halo groups.

“Alkoxy” refers to the group —O-alkyl wherein alkyl is defined herein. Alkoxy includes, by way of example, methoxy, ethoxy, n-propoxy, iso-propoxy, n-butoxy, t-butoxy, sec-butoxy and the like.

“Substituted alkoxy” refers to the group —O-substituted alkyl wherein substituted alkyl is defined herein.

“Acyl” refers to the groups H—C(O)—, alkyl-C(O)— aryl-C(O)—, substituted aryl-C(O)—, heteroaryl-C(O)—, substituted heteroaryl-C(O)—, heterocyclic-C(O)—, and substituted heterocyclic-C(O)—. Acyl includes the “acetyl” group CH₃C(O)—.

“Acylamino” refers to the groups —NR¹⁰C(O)alkyl, —NR¹⁰C(O)-substituted alkyl; —NR¹⁰C(O)aryl, —NR¹⁰C(O)substituted aryl, —NR¹⁰(CO)heteroaryl, —NR¹⁰C(O)substituted heteroaryl, —NR¹⁰C(O)cycloalkyl, —NR¹⁰C(O)substituted cycloalkyl, —NR¹⁰C(O)heterocycloalkyl, and —NR¹⁰C(O)substituted heterocycloalkyl, where R¹⁰ is hydrogen or alkyl.

“Amino” refers to the group —NH₂.

“Amido” refers to the group —C(O)NR¹¹R¹² where R¹¹ and R¹² are independently selected from the group consisting of hydrogen, alkyl, substituted alkyl, aryl, substituted aryl, cycloalkyl, substituted cycloalkyl, heteroaryl, substituted heteroaryl, heterocyclic, and substituted heterocyclic and where R¹¹ and R¹² are optionally joined together with the nitrogen bound thereto to form a heterocyclic or substituted heterocyclic group.

“Amidino” refers to the group —C(═NR¹³)NR¹¹R¹² where R¹¹, R¹², and R¹³ are independently selected from the group consisting of hydrogen, alkyl, substituted alkyl, aryl, substituted aryl, cycloalkyl, substituted cycloalkyl, heteroaryl, substituted heteroaryl, heterocyclic, and substituted heterocyclic and where R¹¹ and R¹² are optionally joined together with the nitrogen bound thereto to form a heterocyclic or substituted heterocyclic group. In one embodiment, the amidino group is —C(═NH)NH₂.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of C₁-C₄ alkyl, hydroxyl, nitro, acyl, acylamino, aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, and C₁-C₄ alkyl-C₁-C₄alkoxy.

“Carboxyl” or “carboxy” refers to —COOH or salts thereof.

“Cyano” refers to the group —CN.

“Cycloalkyl” refers to cyclic alkyl groups of from 3 to 10 and preferably 3 to 7 carbon atoms having single or multiple cyclic rings including fused, bridged, and spiro ring systems. Examples of suitable cycloalkyl groups include, for instance, adamantyl, cyclopropyl, cyclobutyl, cyclopentyl, and cyclooctyl.

“Halo” or “halogen” refers to fluoro, chloro, bromo and iodo.

“Hydroxy” or “hydroxyl” refers to the group —OH.

“Heteroaryl” refers to a heteroaromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Certain non-limiting examples include pyridinyl, pyrrolyl, indolyl, thiophenyl, oxazolyl, thizolyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through a non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” refers to heterocylic groups substituted with 1 to 3 and preferably 1 to 2 substituents selected from the group consisting of hydroxyl, nitro, acyl, acylamino, aryl, substituted aryl, amino, C₁-C₄ alkylamino, di(C₁-C₄ alkyl)amino, cyano, halo, heteroaryl, substituted heteroaryl, thiol, C₁-C₄ thioalkyl, amidino, amido, carboxyl, C₁-C₄ alkoxy, oxo, and C₃-C₇ cycloalkyl.

Examples of heterocycle and heteroaryls include, but are not limited to, azetidine, pyrrole, furan, thiophene, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

“Thiol” refers to the group —SH.

“Alkylthio” refers to the group —S-alkyl wherein alkyl is as defined herein.

Unless indicated otherwise, the nomenclature of substituents that are not explicitly defined herein are arrived at by naming the terminal portion of the functionality followed by the adjacent functionality toward the point of attachment.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to -substituted aryl-(substituted aryl)-substituted aryl.

It is understood that the above definitions are not intended to include impermissible substitution patterns (e.g., methyl substituted with 5 fluoro groups). Such impermissible substitution patterns are well known to the skilled artisan. “Subject” refers to a mammal. The mammal can be a human or non-human animal mammalian organism.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring=N— moiety such as pyrazoles, imidazoles, benzimidazoles, triazoles, and tetrazoles.

“Treating” or “treatment” of a disease or disorder in a subject refers to 1) preventing the disease or disorder from occurring in a subject that is predisposed or does not yet display symptoms of the disease or disorder; 2) inhibiting the disease or disorder or arresting its development; or 3) ameliorating or causing regression of the disease or disorder.

“Pharmaceutically acceptable” refers to a material that is not biologically or otherwise undesirable, e.g., the material may be incorporated into a pharmaceutical formulation administered to a subject without causing and significant undesirable biological effects or interfering in a deleterious manner with any of the other components of the formulation in which it is contained.

“Pharmaceutically acceptable carrier” refers to materials such as solvents, stabilizers, pH-modifiers, tonicity modifiers, adjuvants, binders, diluents and other materials well known to the skilled artisan that are suitable for administration to a subject in combination with the compound or compounds of this invention. The specific carrier selected is predicated in part on the intended route of administration such as rectal, oral, intravenous, parenteral, topical, inhalation, and the like. Such is well within purview of the skilled artisan.

An “effective amount” refers to that amount that results in a desired pharmacological or physiological effect for a specific condition such as an infection. In some cases, an effective amount is that amount sufficient to treat the symptoms of the disease or condition. In some cases, an effective amount is that amount sufficient to partially or completely cure the subject of the disease or condition. In reference to bacterial infections, an effective amount is preferably that amount that reduces the number of bacterial cells, inhibit bacterial growth, and/or kill existing bacteria. In some cases, an effective amount is that amount that is provided to a subject to prevent a bacterial infection when the subject is at risk of such an infection.

### 2. COMPOUNDS OF THE INVENTION

The compounds of this invention are directed to oligopeptide mimetics having from 1 to 9 optionally substituted amino acids and a C-terminal amino acid mimetic of formula I:

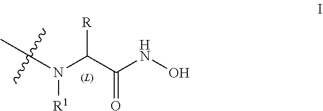

where:

R is selected from hydrogen, optionally substituted aryl, or optionally substituted C₁-C₆ alkyl wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;

R¹ is hydrogen or together with R forms an optionally substituted pyrrolidinyl ring wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;

provided that at least one of the optionally substituted amino acids is an optionally substituted aromatic amino acid selected from the group consisting of optionally substituted phenylglycine, optionally substituted phenylalanine, optionally substituted tyrosine, optionally substituted histidine and optionally substituted tryptophan; and

(L) indicates an L isomer at that stereochemical center.

In one embodiment, this invention provides for an oligopeptide mimetic having from 1 to 9 optionally substituted amino acids and a C-terminal amino acid mimetic of formula II:

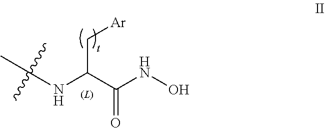

where:

Ar is an optionally substituted phenyl, napthyl, imidazolyl or indolyl group;

t is zero, one, or two;

wherein said optional substitution on said phenyl, napthyl, imidazolyl, or indolyl groups is from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy; and

(L) indicates an L isomer at that stereochemical center;

including pharmaceutically acceptable salts and/or solvates thereof.

In one embodiment, this invention is directed to an oligopeptide mimetic having from 1-9 optionally substituted amino acids and a C-terminal amino acid mimetic of formula III:

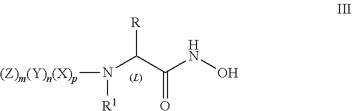

where:

m, n, and pare independently 0 or 1;

X, Y and Z are each independently an L-isomer of an optionally substituted amino acid provided that at least one of R, X, Y and Z is an optionally substituted aromatic amino acid selected from the group consisting of optionally substituted phenylglycine, phenylalanine, optionally substituted histidine, optionally substituted tyrosine, and optionally substituted tryptophan;

wherein said optional substitution on said aromatic amino acids is from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄alkoxy;

R is selected from hydrogen, optionally substituted aryl, or optionally substituted C₁-C₆ alkyl wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and Cᵣ C₄ alkyl-C₁-C₄ alkoxy;

R¹ is hydrogen or together with R forms an optionally substituted pyrrolidinyl ring wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;

(L)_ indicates an L isomer at that stereochemical center;

including pharmaceutically acceptable salts and/or solvates thereof.

In one embodiment, there is provide a compound of formula IV:

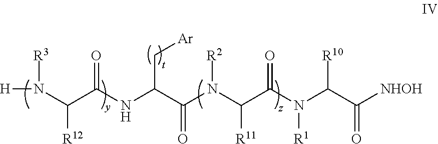

wherein:

R¹⁰, R¹¹, and R¹² are independently an optionally substituted side chain of a non-proline amino acid;

alternatively any one of R¹⁰ and R¹, R¹¹ and R² and/or R¹² and R³ forms an optionally substituted pyrrolidinyl group;

Ar is optionally substituted phenyl, optionally substituted histidyl, or optionally substituted indolyl;

t is 0, 1, or 2; and

y and z are independently 0, 1, 2, 3, 4, 5, 6 or 7 provided that the total of y plus z is no greater than 8;

wherein said optional substitution on said amino acids is from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄alkoxy;

including pharmaceutically acceptable salts and/or solvates thereof.

In one preferred embodiment, the amino acids in the oligopeptides of this invention do not include methionine as such would be compete with the methionine mimetics described herein with the tRNA^{Met} synthesase.

### 3. SYNTHESIS

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of this invention contain one or more chiral centers, such compounds can be prepared or isolated as pure stereoisomers, i.e., as individual enantiomers or d(l) stereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Sigma-Aldrich (Milwaukee, Wis., USA), Bachem (Torrance, Calif., USA), Emka-Chemce or others). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

### Synthesis of Representative Compounds of the Invention

In one general embodiment, the compounds comprise a methionine mimetic coupled to one to seven optionally substituted amino acids wherein at least one of the amino acids is an aromatic amino acid. Specifically, the methionine mimetics employed in the oligopeptides of formula I are readily prepared from the N-protected methyl ester of methionine as shown below:

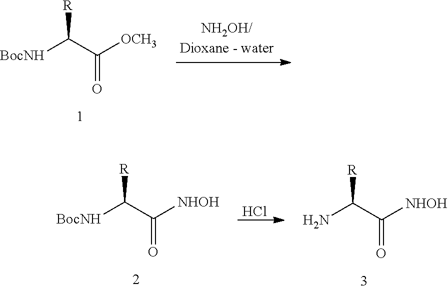

Specifically, the N-Boc protected methyl ester of amino acid (1) is treated with hydroxylamine in a solvent mixture of dioxane and water so as to provide for compound (2). That compound can be isolated or purified by conventional conditions such as chromatography, precipitation, crystallization and the like or, alternatively, used in the next step without isolation and/or purification. Subsequently, the Boc protecting group is removed by conventional conditions such as the addition of an acid such as HCl so as to provide for compound (3). Again, that compound can be isolated or purified by conventional conditions such as chromatography, precipitation, crystallization and the like. Depending upon the side chain of the amino acid used, protection of functional groups such as amino or carboxyl groups will be necessary. As to side chain carboxyl group protection, such protection should be orthogonal to that of the methyl carboxylate. Suitable orthogonal protecting groups are well known in the art.

Also, compounds of type 3 above (with primary amino group on left side of structure) are also part of the invention to the extent they are novel. Also, any compositions, pharmaceutical compositions, bacterial populations, and methods of use (including methods for killing prokaryotic cells and methods for treating subjects) derived from and/or including such compounds of type 3 (with primary amino group on left side of structure) are also part of the invention to the extent they are novel.

Also, compounds derived from Formula II if appropriately reacted to produce a primary amino group on left side of structure are also part of the invention to the extent they are novel. Also, any compositions, pharmaceutical compositions, bacterial populations, and methods of use (including methods for killing prokaryotic cells and methods for treating subjects) derived from and/or including such compounds derived from Formula II if appropriately reacted to produce a primary amino group on left side of structure are also part of the invention to the extent they are novel.

Compound (3) is then coupled to an amino acid chain of from 1 to 7 amino acids using conventional amino acid coupling conditions well known in the art as shown in the following reaction scheme:

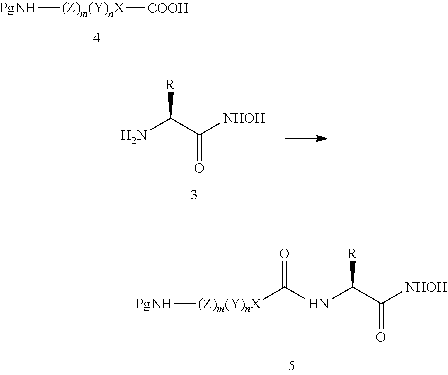

where m, n, R, X, Y and Z are as defined above and Pg is an amino protecting group such as a Cbz group. Upon coupling completion, the resulting compound is isolated and purified as described above. Removal of the amino protecting group provides for the desired oligopeptide.

### 4. FORMULATIONS AND METHODS OF USE

In general, the compounds and compositions of this invention are useful in killing prokaryotic cells such as bacteria. As such, these compounds and compositions are capable of treating bacterial infections in subjects when administered thereto in an effective amount. Examples of bacteria and bacterial infections that are treatable by the compounds and compositions described herein include, without limitation, Escherichia coli, Pseudomonas aeruginosa, Klebsiella pneumonia, Acinetobacter baumannii, Neisseria gonorrhoeae, Haemophilus influenza, Clostridium difficile (C. diff), Enterobacter faecalis, Staphylococcus aureus, Methicillin Resistant Staphylococcus aureus (MRSA), Serratia marcescens, Helicobacter pylori, Saccharomyces cervisiae, Streptococcus thermophiles, Lactococcus lactis, Streptococcus agalactiae, Beta Hemolytic streptococcus, Mycobacterium bovis, Listeria monocytogenes, Peptostreptococcus micros, Fusobacterium nucleaturm, Porphyromonas gingivalis, Salmonella tyrphimurium, and/or Bacciluss subtillus, which may infect, for example, wounds, skin, eyes, ears, nose and/or the GI tract.

In some embodiments, the compounds and compositions of this invention are capable of inhibiting bacterial growth and, accordingly, are useful as bactericidal, antibacterial, and anti-infective agents.

In some embodiments, the compounds and compositions of this invention are capable of inhibiting intracellular bacterial protein synthesis by at least 20%, or by at least 50%, or by at least 75%, or by at least 90%, or by at least 95% or 100% when compared to intracellular bacterial protein synthesis in the absence of the compounds and compositions described herein.

In some embodiments, the compounds and compositions of this invention are capable of intrabacterial inhibition of methionyl-tRNA synthetase by at least 20%, or by at least 50%, or by at least 75%, or by at least 90%, or by at least 95% or 100% when compared to the enzymatic activity in the absence of the compounds and compositions described herein.

In some embodiments, the compounds of this invention are effective when administered to a subject in a therapeutically effective amount. Preferably such amounts range from about 0.1 μg/kg to about 300 mg/kg when administered orally, intravenously, intra-arterially, intraperitoneally, intramuscularly, subcutaneously, intraocularly, rectally, transdermally, intrapulmonarily, and the like. In some embodiments, the amounts so administered more preferably range from about 1 μg/kg to about 40 mg/kg.

In some embodiments, the compounds and compositions are administered topically such as cream, ointment, lotion, and the like. When so applied, the amount of compound employed in such topical formulations ranges from 0.1 mg/mL to about 100 mg/mL.

In all cases, the amount of compound administered to the subject depends upon the weight, age, sex, severity of the condition to be treated and other factors well known to the skilled clinician. In some embodiments, the compounds of this invention can be administered at least once a day, preferably once or twice a day, and in some cases, three or more times a day.

### FORMULATION EXAMPLES

The following are representative pharmaceutical formulations containing a compounds of this invention.

### Formulation Example 1—Tablet Formulation

The following ingredients are mixed intimately and pressed into single scored tablets.

| Ingredient | Quantity per tablet, mg |
|---|---|
| compound of this invention | 400 |
| Cornstarch | 50 |
| croscarmellose sodium | 25 |
| Lactose | 120 |
| magnesium stearate | 5 |

### Formulation Example 2—Capsule Formulation

The following ingredients are mixed intimately and loaded into a hard-shell gelatin capsule.

| Ingredient | Quantity per capsule, mg |
|---|---|
| compound of this invention | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| Flavoring | 0.035 | mL |
| Colorings | 0.5 | mg |
| distilled water | q.s. to 100 | mL |

### Formulation Example 4—Injectable Formulation

The following ingredients are mixed to form an injectable formulation.

| Ingredient | Amount |
|---|---|
| compound of this invention | 0.2 mg-20 mg |
| sodium acetate buffer solution, 0.4M | 2.0 mL |
| HC1 (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of this invention with Witepsol® H-15 (triglycerides of saturated vegetable fatty acid; Riches-Nelson, Inc., New York), and has the following composition:

| Ingredient | Amount |
|---|---|
| Compound of this invention | 500 mg |
| Witepsol ® H-15 | Balance |

The following synthetic and biological examples are offered to illustrate this invention and are not to be construed in any way as limiting the scope of this invention. Unless otherwise stated, all temperatures are in degrees Celsius.

### 5. EXAMPLES

This invention is further understood by reference to the following examples, which are intended to be purely exemplary of this invention. This invention is not limited in scope by the exemplified embodiments, which are intended as illustrations of single aspects of this invention only. Any methods that are functionally equivalent are within the scope of this invention. Various modifications of this invention in addition to those described herein will become apparent to those skilled in the art from the foregoing description and accompanying figures. Such modifications fall within the scope of the appended claims.

In the examples below, the following abbreviations have the following meanings. If an abbreviation is not defined, it has its generally accepted meaning.

calcd=calculated

g=gram

M+H=molecular mass plus proton

mL=milliliter

mmol=millimol

MS=Mass Spectroscopy

N=Normal

μg=microgram

### A. Chemistry

### Example 1: Synthesis of (S)-2-amino-N-hydroxy-4-(methylthio)propanamide hydrochloride (HCl salt. 5-enantioner of I-AA)

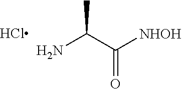

To a solution of (S)-methyl 2-amino-propanoate (30.7 mmol) in dioxane (50 mL) and water (20 mL) at room temperature is added sodium carbonate (5.3 g, 50 mmol) and Boc anhydride (7.96 g, 36.8 mmol). The mixture is stirred overnight at room temperature and then followed by dioxane removal under vacuum. The aqueous layer is extracted with ethyl acetate (3×). The combined organic layers are washed with 1N HCl, brine, and dried over anhydrous sodium sulfate, filtered and concentrated to give a residue. That residue is purified on a silica gel column to give (S)-methyl 2-(2-tert-butoxycarbonylamino)propanoate.

A solution of (S)-methyl-2-(tert-butoxycarbonylamino)propanoate (3.8 mmol) in dioxane (10 mL) and hydroxylamine (50% in water, 10 mL) is stirred at room temperature for 2 days. The solution is diluted with ethyl acetate (200 mL). The organic layer is washed with 1 N HCl, brine, and dried over anhydrous sodium sulfate, filtered and concentrated to give a residue, which is purified on a silica gel column (hexane:ethyl acetate, 1:1 to pure ethyl acetate) to give (S)-tert-butyl 1-(hydroxyamino)-1-oxopropan-2-ylcarbamate.

To solid (S)-tert-butyl 1-(hydroxyamino)-1-oxopropan-2-ylcarbamate (1.25 mmol) is added 4 N HCl in dioxane (2 mL, 8 mmol). The mixture is stirred at room temperature for one hour, and concentrated. The residue is titrated with ether, and dried to provide the title compound.

### Example 2: Synthesis of (S)-2-amino-N-hydroxy-4-(methylthio)butanamide hydrochloride MCI salt, S-enantioner of I-AA)

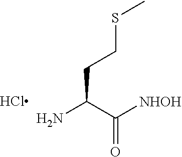

To a solution of (S)-methyl 2-amino-4-(methylthio)butanoate (5 g, 30.7 mmol) in dioxane (50 mL) and water (20 mL) at room temperature was added sodium carbonate (5.3 g, 50 mmol) and Boc anhydride (7.96 g, 36.8 mmol). The mixture was stirred overnight at room temperature followed by dioxane removal under vacuum. The aqueous layer was extracted with ethyl acetate (3×). The combined organic layers were washed with 1N HCl, brine, and dried over anhydrous sodium sulfate, filtered and concentrated to give a residue. That residue was purified on a silica gel column to give (S)-methyl 2-(2-tert-butoxycarbonylamino)-4-(methylthio)butanoate (5.1 g, 63%). MS calcd for (C₁₁H₂₁NO₄S+H)⁺: 264.1; MS found: (M+H)⁺=264.1, 164.1 (minus the t-Boc group).

A solution of (S)-methyl-2-(tert-butoxycarbonylamino)-4-(methylthio)butanoate (1 g, 3.8 mmol) in dioxane (10 mL) and hydroxylamine (50% in water, 10 mL) was stirred at room temperature for 2 days. The solution was diluted with ethyl acetate (200 mL). The organic layer was washed with 1 N HCl, brine, and dried over anhydrous sodium sulfate, filtered and concentrated to give a residue, which was purified on a silica gel column (hexane:ethyl acetate, 1:1 to pure ethyl acetate) to give (S)-tert-butyl 1-(hydroxyamino)-4-(methylthio)-1-oxobutan-2-ylcarbamate (0.33 g, 33%). MS calcd for (C₁₁H₂₀N₂O₄S+H)⁺: 265.1; MS found: (M+H)⁺=266.2, 166.2 (minus the t-Boc group).

To solid (S)-tert-butyl 1-(hydroxyamino)-4-(methylthio)-1-oxobutan-2-ylcarbamate (0.33 g, 1.25 mmol) was added 4 N HCl in dioxane (2 mL, 8 mmol). The mixture was stirred at room temperature for one hour, and concentrated. The residue was titrated with ether, and dried to provide the title compound (S)-2-amino-N-hydroxy-4-(methylthio)butanamide hydrochloride (0.18 g, 80%). MS calcd for (C₅H₁₂N₂O₂S−H)⁺: 163.1; MS found: (M−H)⁺=163.0; ¹NMR (MeOH-d) δ: 3.82, m, 1H; 2.55, m, 2H; 2.12, s, 3H; 1; 2.1, m, 2H.

### Example 3: Synthesis of L-Tryptophan-(S)-2-amino-N-hydroxy-4-(methylthio)butanamide hydrochloride (HCl salt, S-enantioner)

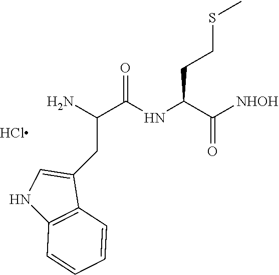

To a solution of (S)-methyl 2-amino-4-(methylthio)butanoate and approximately 1.2 equivalents of Boc-tryptophan in dichloromethane at room temperature was added approximately 1.2 equivalents of diisopropylcarbodiimide and approximately 1.2 equivalents of diisopropylethylamine. The mixture was stirred at room temperature until the reaction was substantially complete, wahed with 1N HCl, brine and dried over sodium sulfate, filtered and concentrated. The title compound was recovered by silica gel chromatography. ¹NMR (MeOH-d) δ: 9.72, m, 1H; 7.68, m, 1H; 7.40, m, 1H; 7.12, m, 4H; 4.44, m, 1H; 4.18, m, 1H; 3.4, m, 1H; 0.3.18, m, 1H; 2.52, m, 2H; 2.1, s, 3H; 2.0, m, 2H;

### Example 4: Synthesis of L-phenylalanine-(S)-2-amino-N-hydroxy-4-(methylthio)butanamide hydrochloride (HCl salt, S-enantioner)

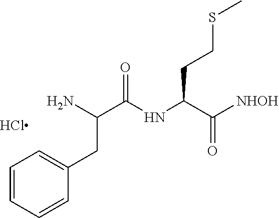

To a solution of (S)-methyl 2-amino-4-(methylthio)butanoate and approximately 1.2 equivalents of Boc-phenylalanine in dichloromethane at room temperature was added approximately 1.2 equivalents of diisopropylcarbodiimide and approximately 1.2 equivalents of diisopropylethylamine. The mixture was stirred at room temperature until the reaction was substantially complete, washed with 1N HCl, brine and dried over sodium sulfate, filtered and concentrated. The title compound was recovered by silica gel chromatography. ¹NMR (MeOH-d) δ: 7.32, bm, 5H; 4.44, m, 1H; 4.15, m, 1H; 3.25, m, 1H; 3.0, m, 1H; 2.52, m, 2H; 2.1, s, 3H; 2.0, m, 2H;

### Example 5: Synthesis of L-tyrosine-(S)-2-amino-N-hydroxy-4-(methylthio)butanamide hydrochloride (HCl salt, S-enantioner)

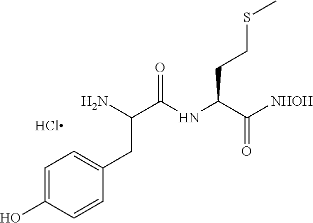

To a solution of (S)-methyl 2-amino-4-(methylthio)butanoate and approximately 1.2 equivalents of Boc-tyrosine in dichloromethane at room temperature was added approximately 1.2 equivalents of diisopropylcarbodiimide and approximately 1.2 equivalents of diisopropylethylamine. The mixture was stirred at room temperature until the reaction was substantially complete, washed with 1N HCl, brine and dried over sodium sulfate, filtered and concentrated. The title compound was recovered by silica gel chromatography. ¹NMR (MeOH-d) δ: 7.1, d, 2H; 6.89, d, 2H; 4.43, m, 1H; 4.07, m, 1H; 3.19, m, 1H; 2.92, m, 1H; 2.52, m, 2H; 2.1, s, 3H; 2.0, m, 2H;

### B. Biology

### Comparative Compounds

The following L,L-dipeptides were tested for their minimum inhibitory concentrations against three different bacterial strains as set forth in the table below. The testing protocol followed conventional methods and identified a MIC value reported as micrograms per milliliter (μg/mL). Each of the dipeptides had the following structure:

TABLE 1

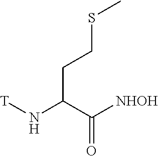

| T | Compound | E. coli | S. aur. | P. aer. |
|---|---|---|---|---|
| Gly | A | 138 | 84 | 647 |
| Ala | B | 158 | 131 | 1040 |
| Val | C | 520 | 416 | 833 |
| Pro | D | 312 | 125 | 270 |
| Lys | E | 312 | 104 | >1660 |
| Glu | F | 554 | 312 | 1660 |

The above results illustrate that of the compounds tested, compound A was most active against E. coli and S. aureus.

### Example 6—Side Chain Substitution of the Methionine Mimetic

Based on the above results, the sidechain of the methionine mimetic was modified to evaluate the effect of such substitution on MIC values. Again, this test used conventional assays to measure the MIC values of each of these modified mimetics. The results of this test are set forth in Table 2 below:

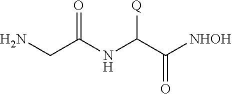

| Q = | Compound | E. coli | S. aur. | P. aer. |
|---|---|---|---|---|
| —CH₂CH₂SCH₃ | A | 138 | 84 | 647 |
| —CH₂CH₂SOCH₃ | G | 123 | 51 | 416 |
| —CH₂CH₂SO₂CH₃ | H | 208 | 104 | 416 |
| —CH₂CH₂CH₂CH₃ | I | 69 | 51 | 520 |
| —CH₂CH₂SCH₂CH₃ | J | 129 | 60 | 554 |
| —CH₂CH₂S(CH₂)₃CH₃ | K | 150 | 60 | 138 |
| —CH₂CH₂SC(CH₃)₃ | L | 207 | 77 | 624 |
| —CH₂SCH₂CH₃ | M | 159 | 104 | 832 |
| —CH₂SC(CH₃)₃ | N | 195 | 77 | 693 |

The date in Table 2 demonstrates that there is flexibility in the side-chain of the methionine mimetic including q equal to 1 or 2, X═S, SO, SO₂ and CH₂.

### Example 7—Zone of Inhibition Test

Compound A was tested against L-phenylalanine-(S)-2-amino-N-hydroxy-4-(methylthio)butanamide hydrochloride (Example 3) in a side-by-side comparison in a conventional zone of inhibition test. In this test, both compounds were compared at various concentrations against E. coli (ATTC 8739) grown in a Petri dish using the antibiotic kanamycin as a control.

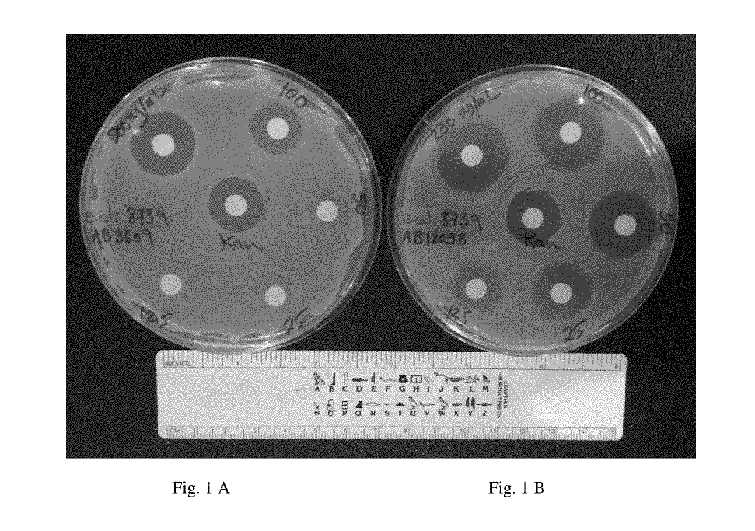

The results are illustrated in FIG. 1A (cmp. A) and FIG. 1B (Ex. 3). As is very clear, cpd. A did not show any noticeable zone of inhibition until the concentration of that compound reached 50 μg and even there, the zone was quite weak.

In contrast thereto, Ex. 3 demonstrated a zone of inhibition at a concentration of 12.5 μg wherein that zone was substantially stronger than the zone of inhibition for cpd. A at 50 gig.

Taken together, this data demonstrates that phenylalanine, and by extension other aromatic amino acids, are significantly more active in killing bacterial cells than compound A when combined with the methionine mimetic. Still further, as the aromatic amino acids facilitate transport across the prokaryotic cell wall and are not involved in inhibiting tRNA^{Met} synthesase, the improved efficacy evidenced by FIG. 1A and FIG. 1B must correlate to the improved intracellular concentration of the methionine mimetic. Still further, the data provided in Table 2 above showing efficacy of other methionine mimetic compounds reasonably correlates to a conclusion that such compounds also will exhibit improved efficacy.

While some embodiments have been illustrated and described, a person with ordinary skill in the art, after reading the foregoing specification, can effect changes, substitutions of equivalents and other types of alterations to the compounds of this invention or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

This invention is also not to be limited in terms of the particular aspects described herein, which are intended as single illustrations of individual aspects of this invention. Many modifications and variations of this invention can be made without departing from its spirit and scope, as will be apparent to those skilled in the art. Functionally equivalent methods within the scope of this invention, in addition to those enumerated herein, will be apparent to those skilled in the art from the foregoing descriptions. Such modifications and variations are intended to fall within the scope of the appended claims. It is to be understood that this invention is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of this invention indicated only by the appended claims, definitions therein and any equivalents thereof.

The embodiments, illustratively described herein, may suitably be practiced in the absence of any element or elements, limitation or limitations, not specifically disclosed herein. Thus, for example, the terms “comprising” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of this invention. This includes the generic description of this invention with a proviso or negative limitation removing any subject matter from the genus, regardless of whether or not the excised material is specifically recited herein.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

What is claimed is:

1. An oligopeptide mimetic having from 1 to 9 optionally substituted amino acids and a C-terminal amino acid mimetic of formula I: 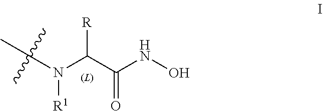
where:
R is selected from hydrogen, optionally substituted aryl, or optionally substituted C₁-C₆ alkyl wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;
R¹ is hydrogen or together with R forms an optionally substituted pyrrolidinyl ring wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;
provided that at least one of the optionally substituted amino acids is an optionally substituted aromatic amino acid selected from the group consisting of optionally substituted phenylglycine, optionally substituted phenylalanine, optionally substituted tyrosine and optionally substituted tryptophan; and
(L) indicates an L isomer at that stereochemical center;
including pharmaceutically acceptable salts and/or solvates thereof.

2. An oligopeptide mimetic having from 1 to 9 optionally substituted amino acids and a C-terminal amino acid mimetic of formula II: 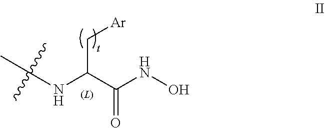
where:
Ar is an optionally substituted phenyl, napthyl, imidazolyl or indolyl group;
t is zero, one, or two;
wherein said optional substitution on said phenyl, napthyl, imidazolyl, or indolyl groups is from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy; and
(L) indicates an L isomer at that stereochemical center;
including pharmaceutically acceptable salts and/or solvates thereof.

3. An oligopeptide mimetic having from 1 to 9 optionally substituted amino acids and a C-terminal amino acid mimetic of formula III: 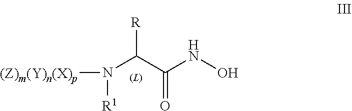
m, n and p are independently 0 or 1;
X, Y and Z are each independently an L-isomer of an optionally substituted amino acid provided that at least one of X, Y and Z is an optionally substituted aromatic amino acid selected from the group consisting of optionally substituted phenylglycine, optionally substituted phenylalanine, optionally substituted tyrosine, optionally substituted histidine, and optionally substituted tryptophan;
wherein said optional substitution on said amino acids is from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄alkoxy;
R is selected from hydrogen, optionally substituted aryl, or optionally substituted C₁-C₆ alkyl wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;
R¹ is hydrogen or together with R forms an optionally substituted pyrrolidinyl ring wherein said optional substitution is with from 1 to 3 substituents selected from the group consisting of C₁-C₄ alkyl, hydroxyl, nitro, acyl, acylamino, aryl, substituted aryl, amino, C₁-C₄ alkylamino, di(C₁-C₄ alkyl)amino, cyano, halo, C₁-C₄ haloalkyl, heteroaryl, substituted heteroaryl, heterocyclic, substituted heterocyclic, thiol, C₁-C₄ thioalkyl, amidino, amido, carboxyl, C₁-C₄ alkoxy, C₃-C₇ cycloalkyl, oxo, and C₁-C₄ alkyl-C₁-C₄ alkoxy;
(L) indicates an L isomer at that stereochemical center;
including pharmaceutically acceptable salts and/or solvates thereof.

4. (canceled)

5. An oligopeptide mimetic according to claim 1,
wherein R is selected from the group consisting of hydrogen, phenyl, (R²⁰)ₐ substituted phenyl, C₁-C₆ alkyl, and (R²¹)_{b}-substituted-(C₁-C₆ alkyl) where R²⁰ is selected from the group consisting of hydroxyl, C₁-C₄ alkyl, C₁-C₄ alkoxy, halo, thiol, amino, nitro, cyano, and carboxy;
R²¹ is selected from the group consisting of hydroxyl, C₁-C₄ alkyl, C₁-C₄ alkoxy, halo, thiol, amino, amido, nitro, cyano, carboxy, aryl, substituted aryl, heteroaryl, substituted heteroaryl, guanidino, substituted guanidino, and C₁-C₄ alkylthiol; and
a and b are integers of from 1 to 3.

6. An oligopeptide mimetic according to claim 1 wherein R is hydrogen, phenyl, benzyl, 4-hydroxyphenylmethylene, 3-indolylmethylene, 5-histidin-1(H)-ylmethylene, methyl, isopropyl, sec-butyl, 1-methylpropyl, hydroxymethyl, isopropyl, sec-butyl, 1-methyl-propyl, hydroxymethyl, aminocarbonylmethyl, aminocarbonylethyl, 1-hydroxyethyl, 2-thiomethoxyethyl, thiomethyl, R is carboxymethyl, carboxyethyl, 4-aminobutyl, or 3-guanadinopropyl.

7.-25. (canceled)

26. An oligopeptide mimetic according to claim 1, wherein R and R¹ are joined to form a pyrrolidinyl ring or are joined to form a hydroxyl or halo substituted pyrrolidinyl ring.

27. (canceled)

28. A pharmaceutical composition comprising a pharmaceutically acceptable carrier and a compound of claim 1.

29. A bacterial population wherein at least a portion of said bacteria comprise a compound of claim 1 within it intracellular space.

30. A method for killing prokaryotic cells which method comprises administering to said cells a compound of claim 1.

31. The method according to claim 30, wherein the prokaryotic cells are bacterial cells.

32. The method according to claim 31, wherein the bacterial cells are E. coli bacteria.

33. A method for treating a subject with a bacterial infection which method comprises administering to the subject an effective amount of an oligopeptide mimetic compound from claim 1.

34. A method for treating a subject with a bacterial infection which method comprises administering to the subject an effective amount of a pharmaceutical composition according to claim 28.

35. A compound according to claim 3 of formula III as well as their salts and/or solvates wherein said compound is selected from the group consisting of compounds 1-23 below:

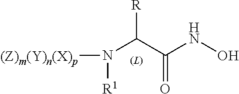

| Comp. | | | | | |
|---|---|---|---|---|---|
| No. | R | R¹ | X | Y | Z |
| 1 | Phenyl | H | Tyrosine | n = 0 | m = 0 |
| 2 | Benzyl | H | Phenylglycine | Alanine | Alanine |
| 3 | 4-Hydroxyphenyl | H | Glycine | Serine | Proline |
| 4 | Methyl | H | Tyrosine | n = 0 | m = 0 |
| 5 | Hydroxyl | H | Tryptophan | Phenylalanine | Serine |
| 6 | 3-Methylpropyl | H | Methionine | Phenylalanine | Cysteine |
| | (leucine side chain) | | | | |
| 7 | Hydroxymethyl | H | Proline | Phenylalanine | m = 0 |
| 8 | Thiomethyl | H | Phenylalanine | n = 0 | m = 0 |
| 9 | R and R¹ = pyrrodinyl | | Tyrosine | Phenylalanine | Glycine |
| 10 | Benzyl | H | Hydroxyglycine | Threonine | Alanine |
| 11 | 4-Hydroxybenzyl | H | Hydroxyglycine | Isoleucine | Aspartic acid |
| 12 | Cysteine | H | 2-amino-3-hydroxy- | Lysine | Phenylalanine |
| | | | 3-phenyl-proprionic | | |
| | | | acid | | |
| 13 | R and R¹ = pyrrodinyl | | Arginine | Cysteine | Tyrosine |
| 14 | Methoxy | H | Phenylalanine | Histidine | Glutamine |
| 15 | 2-Naphthyl | H | Phenylalanine | Valine | Leucine |
| 16 | 3-Indolylmethyl | H | Glycine | Glycine | m = 0 |
| 17 | 3-Indolylmethyl | H | Histidine | Lysine | Glycine |
| 18 | 4-Amino-n-butyl | H | Proline | Tyrosine | Tyrosine |
| 19 | Methoxymethyl | H | Phenylalanine | n = 0 | m = 0 |
| 20 | Ethylthiomethyl | H | 2-amino-3-ethoxy | Phenylalanine | m = 0 |
| | | | proprionic acid | | |
| 21 | R and R¹ = pyrrodinyl | | 2-amino-3-hydroxy- | Valine | Alanine |
| | | | 3-phenyl-proprionic | | |
| | | | acid | | |
| 22 | Phenylalanine | H | 2-amino-3-ethoxy | Leucine | Guanidine |
| | | | proprionic acid | | |
| 23 | Phenylalanine | H | Phenylalanine | Phenylanine | m = 0 |

36. The oligopeptide mimetic of claim 2, wherein the oligopeptide mimetic has 1 or 2 optionally substituted amino acids, Ar is an optionally substituted phenyl or indolyl group; and/or t is zero or one.

37. (canceled)

38. An oligopeptide mimetic according to claim 1, wherein R is hydrogen.

39. An oligopeptide mimetic according to claim 1, wherein R is phenyl.

40. An oligopeptide mimetic according to claim 1, wherein R is benzyl.

41. An oligopeptide mimetic according to claim 1, wherein R is 4-hydroxyphenylmethylene.

42. An oligopeptide mimetic according to claim 1, wherein R is 3-indolylmethylene.
